(19) United States
(12) Patent Application Publication
(10) Pub. No.: US 2019/0022268 A1
(43) Pub. Date: Jan. 24, 2019

(54) PLUG-IN FRAGRANCE DISPENSER WITH TIMED HEATING FUNCTION

(71) Applicant: DONGGUAN YIH TEH ELECTRIC PRODUCTS CO., LTD., Dongguan City (CN)

(72) Inventor: Ming Jen HSIAO, Miaoli County (TW)

(21) Appl. No.: 15/887,232

(22) Filed: Feb. 2, 2018

Related U.S. Application Data
(63) Continuation-in-part of application No. 15/653,362, filed on Jul. 18, 2017.
Application No. 15/653,362 is a continuation-in-part of application No. 15/866,181, filed on Jan. 9, 2018.

(51) Int. Cl. A61L 9/03

(52) U.S. Cl. CPC A61L 9/03; A61L 2209/133; A61L 2209/12; A61L 2209/11

(57) ABSTRACT

A plug-in fragrance dispenser with timed heating function contains: a hollow case, a heat transmission unit, a heating element, a control circuit device, a temperature insulation seat, and a base. The hollow case includes a first opening and a second opening. The temperature insulation seat includes a first orifice and a second orifice, wherein the temperature insulation seat is accommodated in the first opening of the case. The base is housed in the second opening of the case and includes a plug unit. The heat transmission unit is accommodated in the first orifice of the temperature insulation seat. The heating element is mounted in the temperature insulation seat and contacts with a bottom of the heat transmission unit, and the control circuit device is coupled with the base and is electrically connected with the plug unit, wherein the heating element is electrically connected with the control circuit device.

This application is a Continuation-in-Part of application Ser. No 15/653,362, filed Jul. 18, 2017 and Ser. No. 15/866,181, filed Jan. 9, 2018.

### BACKGROUND OF THE INVENTION

### Field of the Invention

The present invention relates to a fragrance dispenser, and more particularly to a plug-in fragrance dispenser which is capable of heating an aroma capsule for a predetermined heating time.

### Description of the Prior Art

A conventional fragrance heating dispenser does not contain a heating time reminder to an aroma capsule, so user cannot know whether the aroma capsule is exhausted or not.

Another conventional fragrance heating dispenser is disclosed in U.S. Pat. Nos. 8,066,420, 8,262,277, and U.S. Pat. No. 8,147,116 and contains a power source, a heating element, essential oil or fragrant wax. However, it is easy to leak the essential oil or fragrant wax out of the conventional fragrance heating dispensers. Moreover, it is troublesome to remove remains of the essential oil or fragrant wax and to wash the fragrance heating dispenser. The fragrance heating dispenser is broken easily because the heating element conducts heat to the electronic components and the case after a period of using time.

The present invention has arisen to mitigate and/or obviate the afore-described disadvantages.

### SUMMARY OF THE INVENTION

The primary aspect of the present invention is to provide a plug-in fragrance dispenser with timed heating function which is capable of isolating heat from the control circuit device and the electronic components so as to prolong service life of the plug-in fragrance dispenser.

Further aspect of the present invention is to provide a plug-in fragrance dispenser with timed heating function which is capable of heating the aroma capsule for a predetermined heating time.

Another aspect of the present invention is to provide a plug-in fragrance dispenser with timed heating function which stops the aromatic substance of the aroma capsule leaking out of the container to cause environmental pollution, wherein the aroma capsule is disposable and is replaceable easily.

To obtain the above aspects, a plug-in fragrance dispenser with timed heating function provided by the present invention contains: a case, a heat transmission unit, a heating element, a control circuit device, a temperature insulation seat, and a base.

The case is hollow and includes a first opening and a second opening, wherein the first opening is defined on a first end of the case, and the second opening is formed on a second end of the case.

The temperature insulation seat includes a first orifice defined on a first end of the temperature insulation seat, and the temperature insulation seat includes a second orifice formed on a second end of the temperature seat, wherein the temperature insulation seat is accommodated in the first opening of the case.

The base is housed in the second opening of the case and includes a plug unit.

The heat transmission unit is accommodated in the first orifice of the temperature insulation seat.

The heating element is mounted in the temperature insulation seat and contacts with a bottom of the heat transmission unit, and the control circuit device is coupled with the base and is electrically connected with the plug unit, wherein the heating element is electrically connected with the control circuit device.

Preferably, the base further includes a first connection member and a second connection member, the first connection member has an abutting plate extending from a top thereof, and the second connection member has a contacting cap extending from a top thereof and has an aperture defined on the contacting cap, the control circuit device is accommodated in the second connection member which connects with the first connection member configured to cover the control circuit device, wherein the abutting plate connects with the contacting cap, and the plug unit couples with the abutting plate via the aperture.

Preferably, the plug unit has a holder and a rotary plug, the holder is connected with the abutting plate, and the rotary plug is rotatably coupled on the holder via the aperture of the contacting cap; the rotary plug is freely rotated toward a desired angle relative to the holder.

Preferably, the first connection member further has a through hole and a hollow post extending from the first connection member and surrounding the through hole, and a free end of the hollow post connects with the second orifice of the temperature insulation seat.

Preferably, the heat transmission unit further includes a support arranged on a bottom thereof, and the support has a fixing orifice defined therein, wherein the plug-in fragrance dispenser with timed heating function further comprises a connection unit, and the heating element abuts against the heat transmission unit, wherein a first end of the connection unit connects on the second connection member, and a second end of the connection unit inserts into the fixing orifice of the support via the second orifice of the temperature insulation seat so as to abut against the heating element.

Preferably, the control circuit device further includes multiple first light emitting elements electrically connected on the control circuit device, and the temperature insulation seat further includes a light guiding element, and the light guiding element extends outward from the first orifice of the temperature insulation seat, wherein the temperature insulation seat is made of transparent or translucent material.

Preferably, the control circuit device has a time setting circuit, and the control circuit device includes multiple second light emitting elements and multiple light guide columns, wherein the multiple second light emitting elements are electrically connected with the control circuit device, a first end of each of the multiple light guide columns connects with each of the multiple second light emitting elements, and a second end of each light guide column extends out of the case so as to guide the lights out of the case from the emitting elements, wherein the time setting circuit of the control circuit device turns on the power source to the heating element after a set time and turns off the power source to the multiple second light emitting elements, and a respective one of the multiple light guide columns of each second light emitting element guides a light signal out of the case so as so indicate a predetermined heating time of the heating element is up.

Preferably, the control circuit device is capable of setting the predetermined heating time of the heating element, and the predetermined heating time is any one of three hours, six hours, and nine hours.

Preferably, the time setting circuit of the control circuit device is configured to store a number of using time of the heating element and to accumulate a total hours of the using time of the heating element up to a predetermined total time, and the predetermined total time is 72 hours.

Preferably, an aroma capsule is placed on the heat transmission unit of the case.

### BRIEF DESCRIPTION OF THE DRAWINGS

FIG. 1 is a perspective view showing the exploded components of a plug-in fragrance dispenser with timed heating function according to a preferred embodiment of the present invention.

FIG. 2 is a perspective view showing the assembly of the plug-in fragrance dispenser with timed heating function according to the preferred embodiment of the present invention.

FIG. 3 is a cross sectional view showing the assembly of the plug-in fragrance dispenser with timed heating function according to the preferred embodiment of the present invention.

### DETAILED DESCRIPTION OF THE PREFERRED EMBODIMENTS

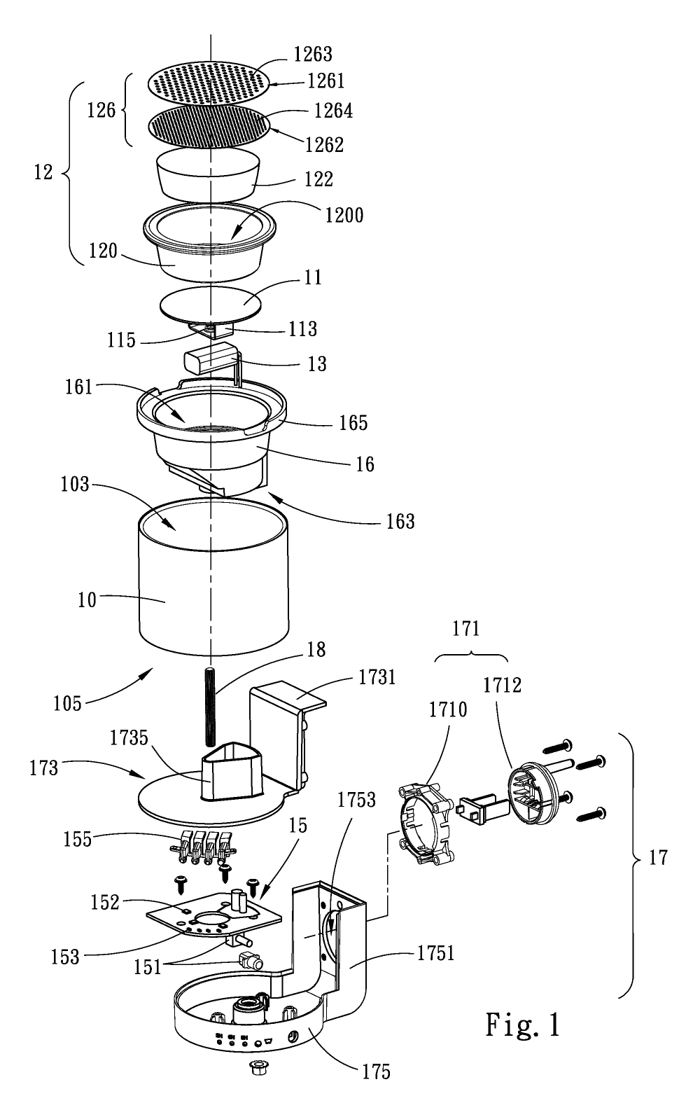

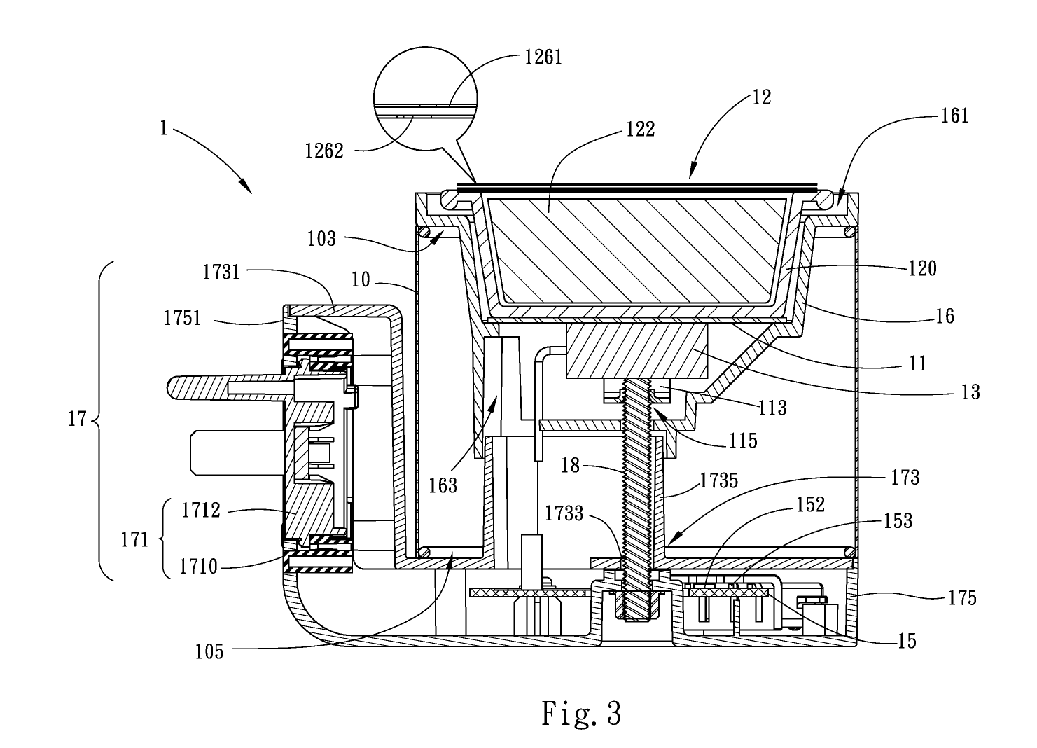

With reference to FIGS. 1-3, a plug-in fragrance dispenser with timed heating function 1 according to a preferred embodiment of the present invention comprises: a case 10, a heat transmission unit 11, a heating element 13, a control circuit device 15, a temperature insulation seat 16, and a base 17.

The case 10 is hollow and includes a first opening 103 and a second opening 105, wherein the first opening 103 is defined on a first end of the case 10, and the second opening 105 is formed on a second end of the case 10.

The temperature insulation seat 16 includes a first orifice 161 defined on a first end thereof and includes a second orifice 163 formed on a second end of the temperature seat 16, wherein the temperature insulation seat 16 is accommodated in the first opening 103 of the case 10. The base 17 is housed in the second opening 105 of the case 10 and includes a plug unit 171, wherein the heat transmission unit 11 is accommodated in the first orifice 161 of the temperature insulation seat 16. The heating element 13 is mounted in the temperature insulation seat 16 and contacts with a bottom of the heat transmission unit 11. The control circuit device 15 is coupled with the base 17 and is electrically connected with the plug unit 171. The heating element 13 is electrically connected with the control circuit device 15, the plug unit 171 is configured to connect with a power supply unit (not shown), and the heating element 13 is configured to heat the heat transmission unit 11, wherein the heating element 13 is any one of a resistor, a thermistor, a cement resistor, and a positive temperature coefficient (PCT) heater.

The control circuit device 15 includes a power switch 151 configured to turn on/off a power source, the temperature insulation seat 16 isolates a high temperature of the heating element 13 so as to stop a heat conductivity of the heating element 13 toward electronic components, the control circuit device 15 or the case 10 of the plug-in fragrance dispenser with timed heating function 1, thus protecting the control circuit device 15 or the electronic components.

Referring to FIGS. 1-3, the base 17 further includes a first connection member 173 and a second connection member 175, wherein the first connection member 173 has an abutting plate 1731 extending from a top thereof, and the second connection member 175 has a contacting cap 1751 extending from a top thereof and has an aperture 1753 defined on the contacting cap 1751. The control circuit device 15 is accommodated in the second connection member 175 which connects with the first connection member 173 configured to cover the control circuit device 15, wherein the abutting plate 1731 connects with the contacting cap 1751, and the plug unit 171 couples with the abutting plate 1731 via the aperture 1753, such that the plug unit 171 connects with a power socket (not shown) so as to supply the power source to the plug-in fragrance dispenser with timed heating function 1.

As shown in FIGS. 1-3, an aroma capsule 12 is mounted in the plug-in fragrance dispenser with timed heating function 1 and faces upward so as to release aroma after being heated.

The plug unit 171 is capable of adjusting its insertion direction in different angles so as to comply with using requirements in various countries. The plug unit 171 has a holder 1710 and a rotary plug 1712, the holder 1710 is connected with the abutting plate 1731, and the rotary plug 1712 is rotatably coupled on the holder 1710 via the aperture 1753 of the contacting cap 1751. The rotary plug 1712 is freely rotated toward a desired angle relative to the holder 1710 so as to comply with the using requirements in various countries. In this embodiment, the rotary plug 1712 is rotated toward the desired angle (such as 270 degrees), wherein the rotary plug 1712 is positioned after being revolved to every 90 degrees.

As illustrated in FIGS. 1-3, the first connection member 173 further has a through hole 1733 and a hollow post 1735 extending from the first connection member 173 and surrounding the through hole 1733, wherein a free end of the hollow post 1735 connects with the second orifice 163 of the temperature insulation seat 16.

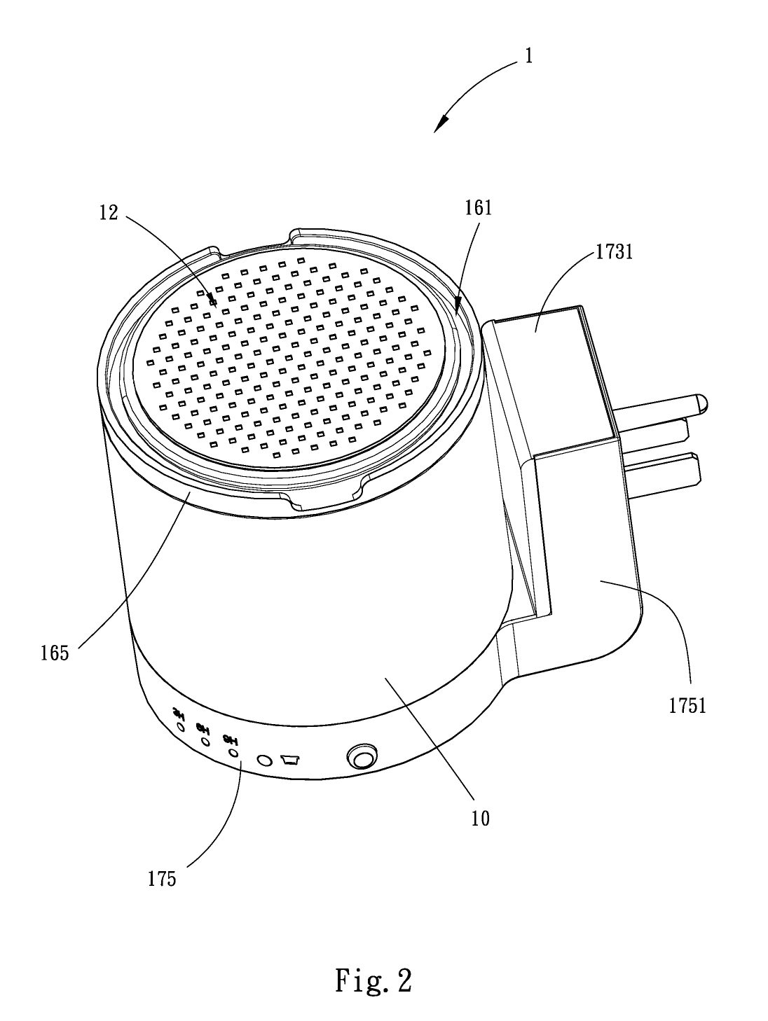

With reference to FIGS. 1 and 2, the heat transmission unit 11 further includes a support 113 arranged on a bottom thereof, and the support 113 has a fixing orifice 115 defined therein. The plug-in fragrance dispenser with timed heating function 1 further comprises a connection unit 18, and the heating element 13 abuts against the heat transmission unit 11, wherein a first end of the connection unit 18 connects on the second connection member 175, and a second end of the connection unit 18 inserts into the fixing orifice 115 of the support 113 via the second orifice 163 of the temperature insulation seat 16 so as to abut against the heating element 13. In this embodiment, the connection unit 18 is a threaded stud or a coupling rod, for example, when the connection unit 18 is the threaded stud, the threaded stud screws in the fixing orifice 115 or the second connection member 175.

The heat transmission unit 11 is a circular disc made of metal so that the heat transmission unit 11 evenly conducts the heat to the aroma capsule 12.

The heat transmission unit 11 is made of thermal conductive material, and the thermal conductive material is any one of metal, ceramics, and glass, hence the heating element 13 conducts the heat to the heat transmission unit 11.

Referring to FIGS. 1-3, the control circuit device 15 further includes multiple first light emitting elements 152 electrically connected on the control circuit device 15.

The temperature insulation seat 16 further includes a light guiding element 165 in a bowl shape, and the light guiding element 165 extends outward from the first orifice 161 of the temperature insulation seat 16, wherein the temperature insulation seat 16 or/and the first connection member 173 are made of transparent or translucent material, and the transparent or the translucent material is any one of plastic, acrylic, and glass. The multiple first light emitting elements 152 emit lights and the lights pass through the first connection member 173, the temperature insulation seat 16 is made of transparent or translucent polypropylene (PP), and the temperature insulation seat 16 receives the lights from the multiple first light emitting elements 152 and guides the lights toward the light guiding element 165 evenly, thus illuminating the lights.

Referring to FIGS. 1-3, the control circuit device 15 has a time setting circuit, and the control circuit device 15 includes multiple second light emitting elements 153 and multiple light guide columns 155, wherein the multiple second light emitting elements 153 are electrically connected with the control circuit device 15, a first end of each of the multiple light guide columns 155 connects with each of the multiple second light emitting elements 153, a second end of each light guide column 155 extends out of the case 10 so as to guide the lights out of the case 10 from the emitting elements 153. The time setting circuit of the control circuit device 15 turns on the power source to the heating element 13 after a set time and turns off the power source to the multiple second light emitting elements 153, and a respective one of the multiple light guide columns 155 of each second light emitting element 153 guides a light signal out of the case 10 so as so indicate a predetermined heating time of the heating element 13 is up, wherein each second light emitting element 153 is an light-emitting diode (LED) or common light emitting elements. Since the light-emitting diode (LED) or the common light emitting elements are prior art, further remarks are omitted.

As shown in FIGS. 1-3, the time setting circuit of the control circuit device 15 is configured to store a number of using time of the heating element 13 and to accumulate a total hours of the using time of the heating element 13 up to a predetermined total time (such as 72 hours), wherein the time setting circuit of the control circuit device 15 turns off the power source to the heating element 13, when the total hours of the using time of the heating element 13 is up to the predetermined total time, and the time setting circuit of the control circuit device 15 sends an instruction to turn on the power source to each second light emitting element 153, thereafter the respective one light guide column 155 of each second light emitting element 153 guides the lights out of the case 10, hence the respective one light guide column 155 sends a light signal to denote that the total hours of the using time of the heating element 13 is up to the predetermined total time so that user learns aromatic substance (such as essential oil or fragrant wax) in the aroma capsule 12 is exhausted.

The respective one light guide column 155 of each second light emitting element 153 is a tube or a post which is made of any one of transparent glass, transparent plastic, and transparent acrylic.

Referring to FIGS. 1-3, in operation, the aroma capsule 12 is placed on the heat transmission unit 11 from the first opening 103 of the case 10, and the control circuit device 15 is electrically connected with the power supply unit (not shown) so as to drive the heating element 13 to heat the heat transmission unit 11, such that the heat transmission unit 11 conducts the heat to the aroma capsule 12, and the aroma capsule 12 disperse fragrance widely. Preferably, the control circuit device 15 is capable of setting the predetermined heating time of the heating element 13 so that the aroma capsule 12 is heated by the heating element 13 for the predetermined heating time, such as three hours, six hours, or nine hours, wherein the predetermined heating time corresponds to each second light emitting element 153 (such as LED). Alternatively, indication symbols (such as 3H, 6H and 9H) of the three hours, the six hours and the nine hours are respectively marked on the case 10 and correspond to the multiple light guide columns 155 respectively. When heating the aroma capsule 12 for the three hours, the control circuit device 15 turns off the power source to the heating element 13, and the time setting circuit sends the instruction to turn on one of the multiple second light emitting elements 153 which corresponds to the 3H, and a light guide column 155 of the one second light emitting element 153 guides the light signal to display outside the case 10, wherein an indication symbol (i.e., 3H) of the three hours is marked on the case 10, such that when the light guide column 155 of the one second light emitting element 153 guides the light signal outside the case 10 so as so display that the predetermined heating time of the heating element 13 reaches the three hours, the plug-in fragrance dispenser with timed heating function 1 stops heating the aroma capsule 12 and controls the control circuit device 15 to record the predetermined heating time. Similarly, other predetermined heating times are set and recorded. After the aroma capsule 12 is heated by the heating element 13 for another predetermined heating times, the control circuit device 15 records an accumulated predetermined heating time.

As shown in FIGS. 1-3, the aroma capsule 12 has a predetermined volume, and the essential oil or the fragrant wax is accommodated in the aroma capsule 12 at the predetermined volume and has an exhausted time. For example, the essential oil or the fragrant wax is heated by the plug-in fragrance dispenser with timed heating function 1 for 72 hours and is exhausted after 72 hours. In other words, the essential oil or the fragrant wax runs out after the aroma capsule 12 is heated for the accumulated predetermined heating time (such as 72 hours which is accumulated from several three hours, six hours, and/or nine hours). Thereafter, the control circuit device 15 sends the instruction to turn off the power source to each second light emitting element 153, and the time setting circuit of the control circuit device 15 sends the instruction to turn on a corresponding second light emitting element 153, then the light guide column 155 of the one second light emitting element 153 guides the light signal to display outside the case 10, such that user learns that the predetermined heating time of the heating element 13 reaches the accumulated predetermined heating time, and the essential oil or the fragrant wax is exhausted, thus replacing a new aroma capsule 12.

The case 10 is made of transparent or translucent material, such as plastic, ceramic with pores, glass with pores, or silicone with pores. Alternatively, the case 10 includes multiple pores so as to guide the lights. Preferably, the multiple first light emitting elements 152 illuminate the lights so that the plug-in fragrance dispenser with timed heating function 1 disperse fragrance and illumines the lights simultaneously.

In addition, the plug-in fragrance dispenser with timed heating function 1 accommodates the aroma capsule 12 on the heat transmission unit 11, and the heating element 13 conducts the heat to the heat transmission unit 1 so as to heat the aroma capsule 12, thus dispersing the fragrance from the aroma capsule 12 efficiently.

As illustrated in FIGS. 1-3, the aroma capsule 12 includes a container 120 having a third opening 1200, the aromatic substance 122 accommodated in the container 120, and an breathable film 126 connected to the third opening 1200 of the container 120 and covering the aromatic substance 122.

Referring to FIGS. 1-2, the breathable film 126 has a fiber fixation layer 1261 and a microporous layer 1262 of excellent moisture permeability. The fiber fixation layer 1261 has multiple first pores 1263 defined therein. The microporous layer 1262 has multiple second pores 1264 formed therein. The fiber fixation layer 1261 and the microporous layer 1262 are connected together so as to form the breathable film 126 which is breathable and waterproof, wherein the microporous layer 1262 is made of thermoplastic elastomer (TPE), and the fiber fixation layer 1261 is made of thermoplastic elastomer (TPE) or polyester. A diameter of the multiple first pores 1263 is more than the multiple second pores 1264 so as to enhance air permeability. The breathable film 126 is connected to the third opening 1200 of the container 120, and the microporous layer 1262 and the multiple second pores 1264 are waterproof and have excellent moisture permeability so as to maintain the fragrance of the aroma capsule 12.

The microporous layer 1262 and the fiber fixation layer 1261 are connected together in a hot pressing manner or in a high frequency manner. The diameter of the multiple first pores 1263 of the fiber fixation layer 1261 is more than the multiple second pores 1264 of the microporous layer 1262 so that heated molecules of the aromatic substance 122 flow out of the multiple first pores 1263 of the fiber fixation layer 1261 through the multiple second pores 1264 of the microporous layer 1262.

The aromatic substance 122 of the aroma capsule 12 is heated in the plug-in fragrance dispenser with timed heating function 1 so as to release the fragrance. As heating the aromatic substance 122, the fiber fixation layer 1261 does not shrink and deform its size, and the microporous layer 1262 does not distort. Furthermore, the heated molecules of the aromatic substance 122 smoothly flow toward atmosphere outside the aroma capsule 12 via the second pores 1264 of the microporous layer 1262 and the first pores 1263 of the fiber fixation layer 1261. Preferably, the aromatic fluid (liquid) does not flow through the second pores 1264, thus enhancing safety of the aroma capsule 12.

The aromatic substance 122 is any one of the fragrant wax, perfume, balsam, sesame oil mixture and the essential oil. In this embodiment, the aromatic substance 122 is the fragrant wax so that the plug-in fragrance dispenser with timed heating function 1 heats the fragrant wax to a melting point, and the heated molecules of the aromatic substance 122 disperse the fragrance. Alternatively, the aromatic substance 122 is the essential oil and is heated to release the heated molecules efficiently.

The container 120 is disposable and is made of polymer. In another embodiment, the container 120 is made of plastics, and the plastic is any one of polyester, poly chloro terephthalate glycol (PCTG), polyethylene terephthalate (PET), propylthiouracil (PTU), and polypropylene (PP). The container 120 does not melt or deform at a predetermined heating temperature and conducts the heat from the plug-in fragrance dispenser with timed heating function 1 to the aromatic substance 122. In another embodiment, the container 120 is made of PCTG so as to conduct the heat from the plug-in fragrance dispenser with timed heating function 1 to the aromatic substance 122, hence the aromatic substance 122 disperses the fragrance. Preferably, the container 120 has excellent ductility and toughness. The container 120 is not damaged easily when being heating by the plug-in fragrance dispenser with timed heating function 1 and efficiently conducts the heat to the aromatic substance 122. The third opening 1200 of the container 120 is connected with the breathable film 126 firmly, i.e., the microporous layer 1262 of the breathable film 126 is connected to the third opening 1200 of the container 120 in a heating manner or by using adhesive.

Referring to FIGS. 1-3, the heated molecules of the aromatic substance 122 smoothly flow toward the atmosphere outside the aroma capsule 12 via the second pores 1264 of the microporous layer 1262 and the first pores 1263 of the fiber fixation layer 1261, so the breathable film 126 is air permeable and does not distort.

Preferably, the container 120 is disposable or is recyclable, and a new aroma capsule 12 is placed on the heat transmission unit 11 after the aromatic substance 122 runs out, hence the user does not touch the aromatic substance 122 and avoids washing the aromatic substance 122. Furthermore, the breathable film 126 stops the aromatic substance 122 leaking out of the container 120 to cause environmental pollution.

While the preferred embodiments of the invention have been set forth for the purpose of disclosure, modifications of the disclosed embodiments of the invention as well as other embodiments thereof may occur to those skilled in the art. The scope of the claims should not be limited by the preferred embodiments set forth in the examples, but should be given the broadest interpretation consistent with the description as a whole.

What is claimed is:

1. A plug-in fragrance dispenser with timed heating function comprising: a case, a heat transmission unit, a heating element, a control circuit device, a temperature insulation seat, and a base;
the case being hollow and including a first opening and a second opening, wherein the first opening is defined on a first end of the case, and the second opening is formed on a second end of the case;
the temperature insulation seat including a first orifice defined on a first end of the temperature insulation seat, and the temperature insulation seat including a second orifice formed on a second end of the temperature seat, wherein the temperature insulation seat is accommodated in the first opening of the case;
the base being housed in the second opening of the case and includes a plug unit;
the heat transmission unit being accommodated in the first orifice of the temperature insulation seat; and
the heating element being mounted in the temperature insulation seat and contacting with a bottom of the heat transmission unit, and the control circuit device being coupled with the base and being electrically connected with the plug unit, wherein the heating element is electrically connected with the control circuit device.

2. The plug-in fragrance dispenser with timed heating function as claimed in claim 1, wherein the base further includes a first connection member and a second connection member, the first connection member has an abutting plate extending from a top thereof, and the second connection member has a contacting cap extending from a top thereof and has an aperture defined on the contacting cap, the control circuit device is accommodated in the second connection member which connects with the first connection member configured to cover the control circuit device, wherein the abutting plate connects with the contacting cap, and the plug unit couples with the abutting plate via the aperture.

3. The plug-in fragrance dispenser with timed heating function as claimed in claim 2, wherein the plug unit has a holder and a rotary plug, the holder is connected with the abutting plate, and the rotary plug is rotatably coupled on the holder via the aperture of the contacting cap; the rotary plug is freely rotated toward a desired angle relative to the holder.

4. The plug-in fragrance dispenser with timed heating function as claimed in claim 3, wherein the first connection member further has a through hole and a hollow post extending from the first connection member and surrounding the through hole, and a free end of the hollow post connects with the second orifice of the temperature insulation seat.

5. The plug-in fragrance dispenser with timed heating function as claimed in claim 4, wherein the heat transmission unit further includes a support arranged on a bottom thereof, and the support has a fixing orifice defined therein, wherein the plug-in fragrance dispenser with timed heating function further comprises a connection unit, and the heating element abuts against the heat transmission unit, wherein a first end of the connection unit connects on the second connection member, and a second end of the connection unit inserts into the fixing orifice of the support via the second orifice of the temperature insulation seat so as to abut against the heating element.

6. The plug-in fragrance dispenser with timed heating function as claimed in claim 3, wherein the control circuit device further includes multiple first light emitting elements electrically connected on the control circuit device, and the temperature insulation seat further includes a light guiding element, and the light guiding element extends outward from the first orifice of the temperature insulation seat, wherein the temperature insulation seat is made of transparent or translucent material.

7. The plug-in fragrance dispenser with timed heating function as claimed in claim 1, wherein the control circuit device has a time setting circuit, and the control circuit device includes multiple second light emitting elements and multiple light guide columns, wherein the multiple second light emitting elements are electrically connected with the control circuit device, a first end of each of the multiple light guide columns connects with each of the multiple second light emitting elements, and a second end of each light guide column extends out of the case so as to guide the lights out of the case from the emitting elements, wherein the time setting circuit of the control circuit device turns on the power source to the heating element after a set time and turns off the power source to the multiple second light emitting elements, and a respective one of the multiple light guide columns of each second light emitting element guides a light signal out of the case so as so indicate a predetermined heating time of the heating element is up.

8. The plug-in fragrance dispenser with timed heating function as claimed in claim 7, wherein the control circuit device is capable of setting the predetermined heating time of the heating element, and the predetermined heating time is any one of three hours, six hours, and nine hours.

9. The plug-in fragrance dispenser with timed heating function as claimed in claim 8, wherein the time setting circuit of the control circuit device is configured to store a number of using time of the heating element and to accumulate a total hours of the using time of the heating element up to a predetermined total time, and the predetermined total time is 72 hours.

10. The plug-in fragrance dispenser with timed heating function as claimed in claim 9, wherein an aroma capsule is placed on the heat transmission unit of the case;
the aroma capsule includes a container having a third opening, an aromatic substance accommodated in the container, and an breathable film connected to the third opening of the container and covering the aromatic substance;
the breathable film has a fiber fixation layer and a microporous layer, the fiber fixation layer has multiple first pores defined therein, the microporous layer has multiple second pores formed therein, and the fiber fixation layer and the microporous layer are connected together so as to form the breathable film.
